(19) United States
(12) Patent Application Publication
(10) Pub. No.: US 2023/0235264 A1
(43) Pub. Date: Jul. 27, 2023

(54) ULTRASONIC DEVICES AND METHODS OF USE

(71) Applicant: CORNING INCORPORATED, Corning, NY (US)

(72) Inventor: Christopher Bowman Horner, Kennebunk, ME (US)

(21) Appl. No.: 18/011,765

(22) PCT Filed: Jun. 21, 2021
(86) PCT No.: PCT/US2021/038190

§ 371 Date: Dec. 20, 2022

Related U.S. Application Data
(60) Provisional application No. 63/043,877, filed on Jun. 25, 2020.

(51) Int. Cl. C12M 1/26; C12M 1/00; C12M 1/24; C12M 3/00

(52) U.S. Cl. CPC C12M 33/08; C12M 29/20; C12M 23/08; C12M 21/08

(57) ABSTRACT

Ultrasonic devices comprise a housing comprising an ultrasonic surface, an ultrasonic transducer, and an ultrasonic horn to provide and focus energy from the ultrasonic transducer to the ultrasonic surface. Methods of removing trapped air bubbles from a cell culture container comprise positioning a cell culture container comprising air bubbles trapped in liquid within microcavity wells of the cell culture container at an ultrasonic surface of an ultrasonic device. Mechanical agitation is generated by the ultrasonic device and applied to the cell culture container to remove the trapped air bubbles from the microcavity wells. Methods of releasing cell aggregates from a cell culture container comprising positioning a cell culture container comprising cell aggregates in microcavity wells at an ultrasonic surface of an ultrasonic device. The cell aggregates are released from the microcavity wells by applying mechanical agitation from the ultrasonic device to a surface of the cell culture container.

This application claims the benefit of priority under 35 U.S.C. § 119 of U.S. Provisional Application Ser. No. 63/043,877 filed on Jun. 25, 2020, the content of which is relied upon and incorporated herein by reference in its entirety.

### TECHNICAL FIELD

This disclosure generally relates to ultrasonic devices and methods of using ultrasonic devices in cell culturing.

### BACKGROUND

The use of spheroids and organoids in three-dimensional (3D) cell culture continues to increase, due to the physiological relevance of cells as structured in living tissues. Spheroid and organoid 3D cell culture are used in many applications, such as tissue engineering, regenerative medicine, and to better understand the pharmacokinetic and pharmacodynamic effects of drugs in preclinical trials, among others.

Commercial spheroid and organoid generation platforms attempt to fulfill the increasing need for generating large quantities of spheroids and organoids for research and other uses. The platforms often involve a substrate having many microcavity wells, and individual spheroids or organoids are to be generated in each well. However, the incorporation of air bubbles in the wells presents problems for spheroid generation. If air bubbles are present prior to seeding the wells with cells, the cells will not be able to settle at the bottom of the wells, thereby preventing homogenous distribution and aggregation forming of spheroids or organoids.

### SUMMARY

Ultrasonic devices according to the invention allow for mechanical removal of trapped air bubbles in a microcavity substrate of a cell culture container, thereby avoiding the techniques of manual agitation or chemical treatment previously used to remove trapped air bubbles. Manual agitation may include tapping or bumping the substrate against a work surface to dislodge the bubbles, but such practice leads to breakage and loss of vessel integrity. Chemical treatment methods to remove air bubbles may include functionalizing the surface of the substrate to increase wettability, pre-wetting the surface with a solvent possessing a lower wetting angle such as ethanol, flushing the system at high flow rates, and coating the surface with a polysaccharide to allow dissolution across the surfaces of the substrate. However, chemical treatment methods require additional process steps and special packaging for the treated substrates.

Devices of the invention use a piezoelectric ultrasonic transducer that vibrates at a consistent frequency. The ultrasonic vibration results in cavitation events, where subjecting the trapped air bubbles to a frequency of sound allows the bubbles to expand and contract, thereby resulting in removal of the bubbles from the microcavity wells in a cell culture container. Thus, the mechanical vibration allows for removal of trapped air bubbles in the microcavity wells without adding complex process steps, requiring special packaging, or causing breakage of the cell culture container. By providing an ultrasonic device having a small footprint, the device can be used in a sterile cell culture hood and can minimize process steps and simplify the user experience by saving time and increasing efficiency of the spheroid generation process.

According to an aspect of the invention, an ultrasonic device comprises a housing comprising an ultrasonic surface; an ultrasonic transducer disposed within the housing; and an ultrasonic horn to provide and focus energy from the ultrasonic transducer to the ultrasonic surface.

The ultrasonic transducer may be an ultrasonic piezoelectric transducer. The ultrasonic frequency of the ultrasonic device may be in a range of about 25kHz to about 100kHz. In some embodiments, the ultrasonic frequency may be about 40 kHz. The ultrasonic device may further comprise an ultrasonic power driver board. The ultrasonic device may provide continuous vibration. The ultrasonic device may provide pulsed vibration.

The ultrasonic surface may comprise an aperture through which a face of the ultrasonic horn protrudes. The device may further comprise a power switch is disposed on a surface of the housing. The device may further comprise indicator lights are disposed on a surface of the housing. The device may further comprise activation switches on the ultrasonic surface. The ultrasonic device may be portable.

The ultrasonic surface may be capable of receiving a cell culture container. The cell culture container may comprise a microcavity plate, microcavity flask, or multilayered microcavity cell culture vessel. In some embodiments, the cell culture container is a microcavity flask and comprises a T-25 flask, a T-75 flask, a T-175 flask, or a T-225 flask. In some embodiments, the cell culture container is a microcavity plate and comprises a 96-well spheroid microplate, a 384-well spheroid microplate, or a 1536-well spheroid microplate. In some embodiments, the cell culture container is a multilayered microcavity cell culture vessel and comprises a CellSTACK® vessel (manufactured by Corning Incorporated, Corning, N.Y.), HYPERFlask® vessel (manufactured by Corning Incorporated, Corning, N.Y.), or HYPERStack® vessel (manufactured by Corning Incorporated, Corning, N.Y.).

According to an aspect of the invention, methods of removing trapped air bubbles from a cell culture container comprise positioning a cell culture container at an ultrasonic surface of an ultrasonic device, the cell culture container comprising air bubbles trapped in liquid within microcavity wells of the cell culture container; and generating mechanical agitation at the ultrasonic surface to remove the trapped air bubbles from the microcavity wells.

The mechanical agitation may be a pulsed vibration. The mechanical agitation may be a continuous vibration. The mechanical agitation generated may comprise using an ultrasonic horn to provide and focus ultrasonic energy from an ultrasonic transducer. The mechanical agitation may be applied to the cell culture container for increments of about 15 seconds or less.

The mechanical agitation may be applied to the cell culture container by holding the cell culture container against the ultrasonic horn at the ultrasonic surface. The cell culture container may be a microcavity plate, microcavity flask, or multilayered microcavity cell culture vessel. The cell culture container may comprise a microcavity substrate for generating spheroids or organoids.

According to an aspect of the invention, methods of releasing cell aggregates from a cell culture container comprise positioning a cell culture container at an ultrasonic surface of an ultrasonic device, the cell culture container comprising cell aggregates in microcavity wells; and releasing the cell aggregates from the microcavity wells by applying mechanical agitation from the ultrasonic device to a surface of the cell culture container.

The mechanical agitation may be applied to the cell culture container in increments of 15 seconds or less. The mechanical agitation may be applied to the cell culture container continuously. The mechanical agitation may be applied to the cell culture container in pulses. The cell culture container may comprise a microcavity plate, microcavity flask, or multilayered microcavity cell culture vessel. The cell aggregates may be spheroids or organoids. The surface of the cell culture container may be a bottom surface.

According to an aspect of the invention, a kit may comprise an ultrasonic device and a cell culture container comprising a microcavity substrate for generating spheroids or organoids.

Further scope of the applicability of the described devices, methods, and kits will become apparent from the following detailed description, claims, and drawings. The detailed description and specific examples are given by way of illustration only, since various changes and modifications within the spirit and scope of the description will become apparent to those skilled in the art.

### BRIEF DESCRIPTION OF THE DRAWINGS

A further understanding of the nature and advantages of the present disclosure may be realized by reference to the following drawings. In the appended figures, similar components or features may have the same reference label.

FIG. 1 is a perspective view of a device according to an embodiment of the invention.

FIG. 2 is a perspective view of a device according to an embodiment of the invention.

FIG. 3 is a perspective view of a device according to an embodiment of the invention.

FIG. 4 is an exploded view of a device according to an embodiment of the invention.

FIG. 5 shows a method according to an embodiment of the invention.

FIG. 6 shows a cell culture container before (A) and after (B) applying a method according to an embodiment of the invention.

### DETAILED DESCRIPTION

Due to the inherent hydrophobic nature of bulk spheroid microcavity substrate geometry, the hydrogen bonding of water molecules in aqueous solutions creates a high surface tension. That surface tension results in liquid spanning across the top of the microcavities, rather than entering them, thereby preventing wetting of the microcavity wells. Conventional techniques for solving the surface tension problem include chemical treatments to pre-wet the wells or to apply a coating. For example, the wells may be pre-wetted with a gradation of non-polar solvents, such as ethanol, to more polar liquids such as water, with mixtures of each in between. As another example, when coating the substrate in glucose, the affinity for water to the polar molecule allows for better wetting and filling of the microcavity wells. However, both techniques are chemical treatments, which require additional handling and treatment steps prior to cell culture.

In contrast, devices and methods of the invention overcome surface tension using ultrasonic waves. The invention therefore provides a mechanical method, rather than a chemical treatment, which can be used to assist in wetting out the microcavity wells. The devices remove trapped air from the bottom of the microcavity wells using a mechanical agitation rather than a chemical or surface modification. In some embodiments of the invention, a kit is provided that comprises an ultrasonic device accompanying a cell culture container or a bulk spheroid microcavity substrate. Devices and methods of the invention simplify the manufacturing process for microcavity substrates or vessels. By using mechanical agitation, the microcavity substrates no longer require chemical coating steps, such as a polysaccharide coating step. Because the chemical coating is no longer required, the need for special packaging for the microcavity substrate is also eliminated.

The devices and methods of the invention use cavitation of gas bubbles in liquids in response to ultrasonic waves to remove the trapped air bubbles from the bottom of microcavity wells. In an embodiment, a piezoelectric transducer vibrates at a determined frequency when an AC voltage waveform is applied, and the transducer converts electrical energy into mechanical energy. In certain embodiments, devices of the invention comprise ultrasonic piezoelectric transducers operating at frequencies ranging from about 20 kHz upwards to GHz frequencies. When air bubbles or gas bubbles are subjected to high frequency vibration, the change in wave pressure rapidly expands and contracts the bubbles contained within a fluid until the bubbles collapse, thereby releasing a shock wave known as cavitation. By subjecting the microcavity substrate to an ultrasonic frequency, devices of the invention allow the air bubbles to be removed from the bottom of the microcavity wells by a mechanical agitation instead of traditionally used chemical modifications.

Devices and methods of the invention also simplify processing of microcavity substrates for spheroid formation. Devices of the invention generate consistent, predictable mechanical agitation. When the mechanical agitation is applied to a microcavity substrate surface, the need for manual “tapping” or “banging” of the cell culture container on a work surface, such as a laboratory bench, to remove trapped bubbles is eliminated. The size and small footprint of the device also allow for the ultrasonic device to be used within a cell culture hood. Therefore, the device size eliminates the need to remove the substrate from a cell culture hood, such as a Class II cell culture hood, in order to use a benchtop centrifuge or other method to remove air bubbles during the cell culture process and seeding of the cells in the cell culture container. By allowing the air bubbles to be removed within the cell culture hood, the invention shortens the process and handling time while keeping the microcavity substrate within the sterile environment of the cell culture hood.

Devices and methods of the invention also prove useful in later stages of the spheroid or organoid generation process. The invention may be used to dislodge spheroids or organoids attached to the microcavity substrate after an extended period of culture. Providing mechanical agitation using devices of the invention allows for dislodging the spheroids or organoids without destroying the spheroids or organoids or damaging the cells contained within.

Ultrasonic devices of the invention comprise a housing comprising an ultrasonic surface, an ultrasonic transducer disposed within the housing, and an ultrasonic horn to provide and focus energy from the ultrasonic transducer to the ultrasonic surface. The ultrasonic transducer may be a piezoelectric ultrasonic transducer. In some embodiments, the device also comprises activation switches, a power switch, settings switch, indicator light, or a combination thereof. In some embodiments, a kit is provided that comprises an ultrasonic device according to the invention and a bulk microcavity substrate for generating spheroids or organoids.

Devices of the invention further comprise a power source and an ultrasonic power driver board or control board to amplify power. The power source may be any suitable power source, such as a battery or electrical outlet. The device may comprise an AC input cable and plug or battery input. Any suitable controller may be used. For example, the controller may comprise a control board, power amplification module, or a combination thereof. Activation switches may be arranged around the perimeter of the face of the ultrasonic horn. A power switch may be provided to power down the device to save energy and also to prevent undesired operation when the device is not is use.

A surface of the housing may comprise an ultrasonic surface. The ultrasonic surface may be a topmost surface of the device. The ultrasonic surface may comprise an aperture through which a face of the ultrasonic horn protrudes. The ultrasonic surface is capable of receiving a cell culture container. In some embodiments, the cell culture container comprises a microcavity plate, microcavity flask, or multilayered microcavity cell culture vessel. In an embodiment, the ultrasonic device has a small footprint, thereby allowing the device to be used within a cell culture hood without taking up valuable space under the hood. In an embodiment, the device is small, modular, and has dimensions suitable for use in a cell culture hood. In some embodiments, the device is shaped like a 6-inch cube. The device may comprise sealed internal components, thereby allowing the device to be easily wiped down or disinfected for use in the sterile cell culture hood. In some embodiments, the device is portable.

The housing may be formed from any suitable material that provides a rigid structure. For example, the housing may be formed from a rigid plastic material or other rigid non-conductive material. In some embodiments, at least a portion of the ultrasonic horn protrudes from the ultrasonic surface of the housing. The portion of the ultrasonic horn that protrudes from the ultrasonic surface may be a face of the ultrasonic horn.

Devices and methods of the invention may use any suitable ultrasonic frequency and any suitable ultrasonic transducer which operates at the desired ultrasonic frequency. In some embodiments, the ultrasonic frequency for cavitation is in a range of about 25 kHz to about 100 kHz. In some embodiments, the ultrasonic frequency is about 40 kHz. The ultrasonic transducer may be a piezoelectric ultrasonic transducer. The ultrasonic transducer may be a 40 kHz 60 W ultrasonic transducer. In some embodiments, the ultrasonic frequency is in a range of about 25 kHz to about 100 kHz. In some embodiments, the ultrasonic frequency is selected from 25 kHz, 40 kHz, and 80 kHz. In preferred embodiments, the ultrasonic frequency is 40 kHz. Preferably, the ultrasonic frequency is greater than the range of human hearing, which is up to about 20 kHz. In an embodiment, the ultrasonic transducer may be a 40kHz piezoelectric ultrasonic transducer manufactured by KEMET Electronics Corporation (Fort Lauderdale, Fla.) or a 40 kHz piezoelectric ultrasonic air transducer manufactured by Steiner & Martins, Inc. (Davenport, Fla.). It should be understood that other ultrasonic transducers are contemplated and possible.

The horn may be any suitable ultrasonic horn that can focus the ultrasonic energy emitted from the ultrasonic transducer. The ultrasonic horn may be any suitable size and shape and formed from any suitable material. In an embodiment, the ultrasonic horn is a metal, cylindrical ultrasonic horn. The ultrasonic horn may be manufactured from any suitable material such as aluminum, titanium, or steel. As an example, the ultrasonic horn may be shaped like a cylinder or a rectangular block. In an embodiment, the ultrasonic horn may be a cylindrical aluminum ultrasonic horn such as those manufactured by Branson Ultrasonics Corp. (Danbury, Conn.) or Sonitek Corporation (Milford, Conn.). It should be understood that other ultrasonic horns are contemplated and possible.

As described herein, devices of the invention may be used to remove trapped air bubbles from the bottom of microcavity wells to aid in equal distribution of cell seeding for bulk spheroid formation. Devices of the invention may also be used to dislodge cells from microcavity wells.

In some embodiments, devices and methods of the invention are used with cell culture containers or bulk spheroid microcavity substrates to “wet-out” or fully saturate the microcavity wells in the cell culture container with an aqueous solution. By using an ultrasonic transducer as a consistent, predictable means of mechanical agitation, trapped air bubbles can be removed from an array of microcavity wells, thereby “wetting-out” the microcavity wells. “Wetting-out” the microcavity wells allows for homogenous cell seeding and formation of cell spheroids across the array of microcavities for downstream processing.

In some embodiments, devices and methods of the invention are used to dislodge spheroids or organoids from microcavity wells. Spheroids cultured for an extended period of time may be lodged tightly in microcavity wells, such as the micron-scale wells of bulk spheroid microcavity substrates or cell culture containers. Previously used methods of removing aggregated cells, such as spheroids or organoids, from the microcavity wells include manual scraping or vigorous rinsing, which may lead to damaged spheroids or organoids. Using an ultrasonic device according to the invention provides a consistent and predictable means of mechanical agitation, which allows for easier removal of spheroids cultured for extended time periods compared to the manual or chemical processes previously used.

In some embodiments, devices of the invention are suitable for use with cell culture containers such as microcavity substrate products. Nonlimiting examples of microcavity substrate products include T-25 flasks, T-75 Flasks, open well plates, and microcavity cell culture plates. Nonlimiting examples of microplate products include EZSPHERE (by Nacalai USA), AGGREWELL (by STEMCELL Technologies), SPHERA (by Nunclon), CELLSTAR (by Greiner Bio-One), CELLCARRIER (by PerkinElmer), and NANOCULTURE (by MBL International), 96-well spheroid microplates (Corning Incorporated, Corning, N.Y.), 384-well spheroid microplates (Corning Incorporated, Corning, N.Y.), 1536-well spheroid microplates (Corning Incorporated, Corning, N.Y.), and ELPLASIA plates (by Corning Incorporated, Corning, N.Y.). In some embodiments, the cell culture container comprises a microcavity plate, microcavity flask, or multilayered microcavity cell culture vessel. In some embodiments, the cell culture container is a microcavity flask and comprises a T-25 flask, a T-75 flask, a T-175 flask, or a T-225 flask. In some embodiments, the cell culture container is a microcavity plate and comprises a 96-well spheroid microplate, a 384-well spheroid microplate, or a 1536-well spheroid microplate. In some embodiments, the cell culture container is a multilayered microcavity cell culture vessel and comprises a CellSTACK® vessel (manufactured by Corning Incorporated, Corning, N.Y.), HYPERFlask® vessel (manufactured by Corning Incorporated, Corning, N.Y.), or HYPERStack® vessel (manufactured by Corning Incorporated, Corning, N.Y.). It should be understood that other cell culture containers are contemplated and possible.

When the device is powered on (in devices with a power switch, the power switch is turned to an ON position and the power source is providing power to the device), the device is in a standby mode until a cell culture container is placed onto a face of the ultrasonic horn at the ultrasonic surface of the device. When the cell culture container is placed on the face of the ultrasonic horn, the ultrasonic transducer is powered on. In devices with activation switches, placing the cell culture container on the ultrasonic surface and the face of the ultrasonic horn may also include triggering activation switches that power on the ultrasonic transducer. When powered on, the ultrasonic transducer vibrates the bulk spheroid microcavity surface, thereby dislodging any trapped air bubbles (or trapped spheroids) within the microcavity wells of the cell culture container. The mechanical action of the vibration is harsh enough to remove any trapped air bubbles, but gentle enough to avoid catastrophic failure to the vessel or substrate.

Depending on the stage progression of the cell culture process, the vibration may be used for cell seeding or for cell harvesting. For cell seeding, the vibration is used to dislodge any trapped air bubbles through cavitation within the microcavity wells to allow for spheroid or organoid generation. For cell harvesting, the vibration and subsequent cavitation is used to dislodge cells from the microcavity wells for harvesting of the generated spheroids or organoids.

Different settings of the device, such as continuous or pulsed vibration, may be provided. In some embodiments, the device provides continuous vibration. In some embodiments, the device provides pulsed vibration. The pulsed vibration may be a pulsed, intermittent vibration. The device may provide mechanical agitation or vibration in increments of about 15 seconds or less. In some embodiments, the pulsing occurs in increments of about 15 seconds or less. Pulsed vibration may be provided by alternating the ON-OFF sequences or impulses of the ultrasonic transducer or by programming timed intervals of ultrasonic vibration. The pulsing allows for more effective clearing of trapped bubbles from the microcavities and limits the amount of heat and sound generated by operation of the device, which thereby prolongs the life of the transducer.

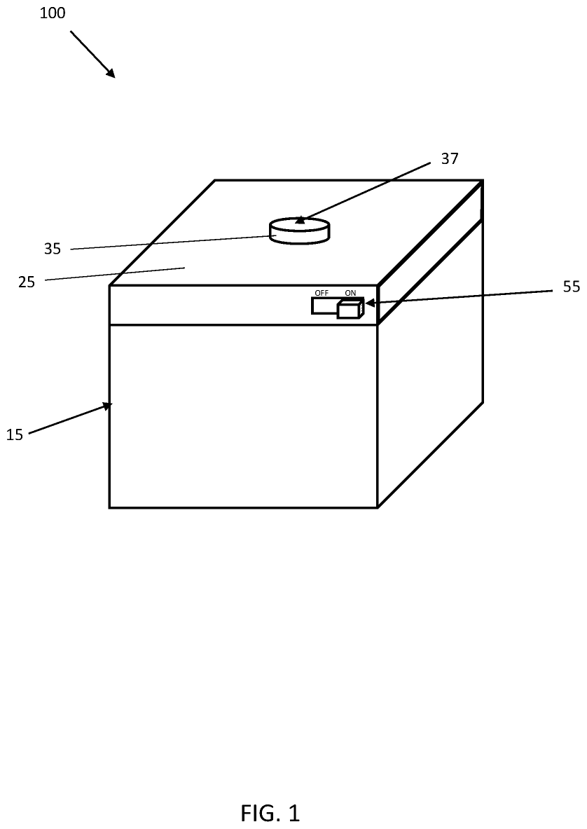

FIG. 1 shows an ultrasonic device 100 according to an embodiment of the invention. The ultrasonic device 100 comprises a housing 15 comprising an ultrasonic surface 25, an ultrasonic transducer disposed within the housing 15, and an ultrasonic horn 35 to provide and focus energy from the ultrasonic transducer to the ultrasonic surface 25. The ultrasonic horn 35 has a face 37 protruding from the ultrasonic surface 25. A power switch 55 that has OFF-ON modes is provided on a front surface of the ultrasonic device 100.

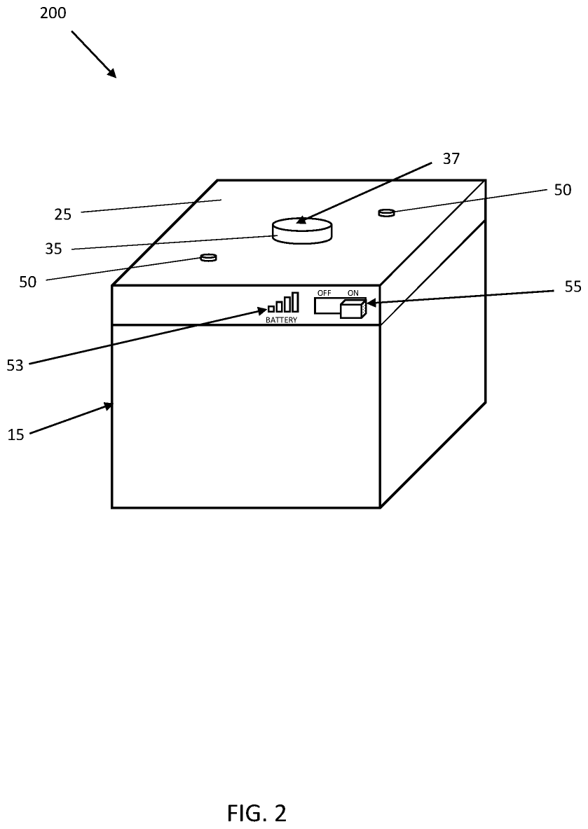

FIG. 2 shows an ultrasonic device 200 according to an embodiment of the invention, wherein the device 200 further comprises activation switches 50 at a perimeter of the face 37 of the ultrasonic horn 35 on the ultrasonic surface 25. Any suitable activation switches or buttons may be used. For example, buttons may be used that depress when a cell culture container is pressed down on the buttons, thereby activating the ultrasonic transducer. An indicator light 53, such as a light indicating battery power level of the device is shown on a front surface of the ultrasonic device.

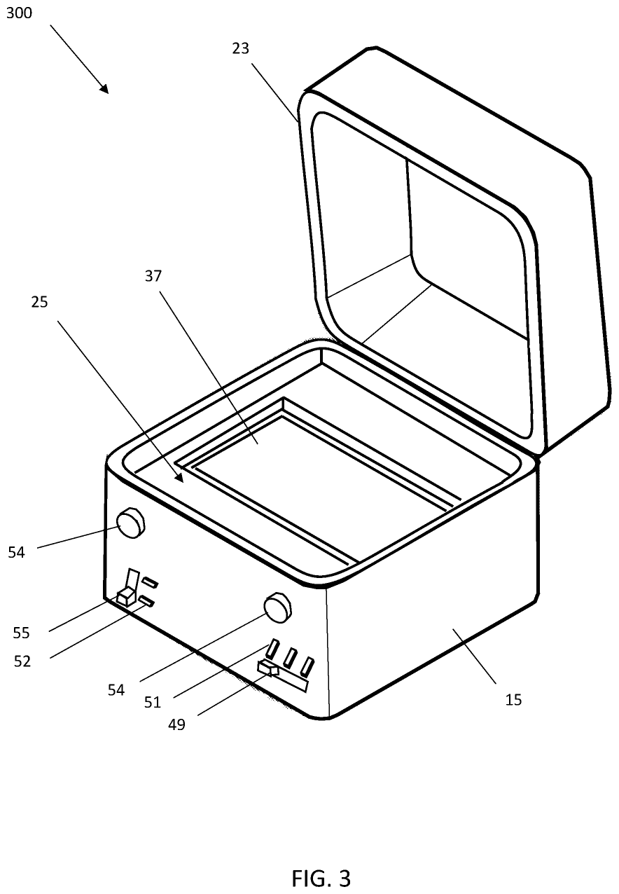

FIG. 3 shows an ultrasonic device 300 according to an embodiment of the invention. The device 300 comprises a rigid housing 15 with an ultrasonic surface 25. A lid 23 that covers the ultrasonic surface 25 is also provided. The lid may be any suitable lid, such as a hinged lid (shown) or a lid with a clasp or an interlocking lid. Other types of lids are contemplated. The ultrasonic surface 25 may be a flat surface, such as a tray, that is suitable for receiving cell culture containers of various sizes and dimensions. An ultrasonic horn face 37 protrudes from the ultrasonic surface 25 of the housing 15. A rectangular shaped ultrasonic horn face 37 is shown.

A power switch 55 is provided on a front surface of the ultrasonic device 300. In some embodiments, the power switch has an ON position and an OFF position. When in the ON position, the ultrasonic source is activated and in standby mode, and the device will provide mechanical agitation when an item contacts the ultrasonic surface. When the power switch is in the OFF position, the ultrasonic source is not active. A setting switch 49 is provided on a front surface of the ultrasonic device 300. The setting switch allows for different settings of the device to be activated. Example settings include pulsed agitation, continuous agitation, and timing settings. Indicator lights 51, 52, and 54 are provided on a front surface of the ultrasonic device 300. The indicator lights may be associated with the power switch, the setting switch, and the ultrasonic surface. For example, indicator lights 51 designate the mode of the setting switch 49, indicator lights 52 designate the mode of the power switch 55, and indicator lights 54 designate whether the ultrasonic transducer is activated and/or providing mechanical vibration to the ultrasonic surface 25.

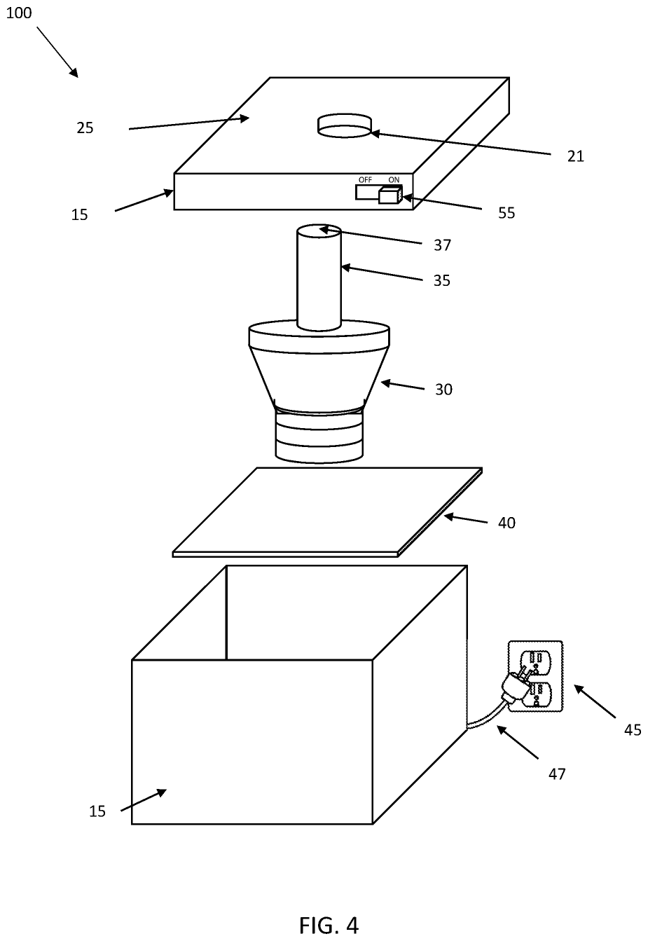

FIG. 4 shows an exploded view of ultrasonic device 100. The device 100 comprises a housing 15. In some embodiments, the housing 15 may be shaped like a cube, as shown. The housing may be a non-conductive material, such as a rigid plastic housing. Disposed within the housing 15 are a controller or control board 40, an ultrasonic transducer 30, an ultrasonic horn 35 having a face 37, and insulating material (not shown). The controller may be any suitable controller, such as an ultrasonic power driver board. A top surface of the housing 15 serves as an ultrasonic surface 25. The ultrasonic surface 25 comprises an aperture 21 through which the face 37 of the ultrasonic horn 35 protrudes. The power source 55 shown is an electrical outlet. An electrical cord and plug 47 provide electrical power from the power source 55 to the device 100 when the power switch 55 is turned to an ON position. The control board 40 and ultrasonic transducer 30 convert the electrical power to ultrasonic power in the form of mechanical agitation, which is focused by the ultrasonic horn 35 and provided to a cell culture container placed flush with the face 37 of the ultrasonic horn 35 at the ultrasonic surface 25.

Methods of removing trapped air bubbles from a cell culture container are provided. According to an aspect of the invention, methods of removing trapped air bubbles from a cell culture container comprise positioning a cell culture container at an ultrasonic surface of an ultrasonic device, the cell culture container comprising air bubbles trapped in liquid within microcavity wells of the cell culture container; and generating mechanical agitation at the ultrasonic surface to remove the trapped air bubbles from the microcavity wells.

The mechanical agitation may be a pulsed vibration. The mechanical agitation may be a continuous vibration. The mechanical agitation generated may comprise using an ultrasonic horn to provide and focus ultrasonic energy from an ultrasonic transducer. The mechanical agitation may be applied to the cell culture container by holding the cell culture container against the ultrasonic horn at the ultrasonic surface. The mechanical agitation may be applied to the cell culture container for increments of about 15 seconds or less.

In some embodiments, a face of the ultrasonic surface protrudes from the ultrasonic surface. In some embodiments, the cell culture container is positioned by holding the cell culture container flush against the face of the ultrasonic horn to apply ultrasonic energy in the form of mechanical agitation to the cell culture container, thereby removing trapped air bubbles. In some embodiments, a bottom surface of the cell culture container is held flush against the ultrasonic horn. In some embodiments, the cell culture container is manually manipulated by a user so that different portions of the bottom surface of the cell culture container are flush with the face of the ultrasonic horn.

In some embodiments, the cell culture container is a microcavity substrate or a bulk microcavity substrate for generating spheroids or organoids. In some embodiments, the cell culture container comprises a microcavity plate, microcavity flask, or multilayered microcavity cell culture vessel. In some embodiments, the cell culture container is a microcavity flask and comprises a T-25 flask, a T-75 flask, a T-175 flask, or a T-225 flask. In some embodiments, the cell culture container is a microcavity plate and comprises a 96-well spheroid microplate, a 384-well spheroid microplate, or a 1536-well spheroid microplate. In some embodiments, the cell culture container is a multilayered microcavity cell culture vessel and comprises a CellSTACK® vessel (manufactured by Corning Incorporated, Corning, N.Y.), HYPERFlask® vessel (manufactured by Corning Incorporated, Corning, N.Y.), or HYPERStack® vessel (manufactured by Corning Incorporated, Corning, N.Y.). It should be understood that other cell culture containers are contemplated and possible.

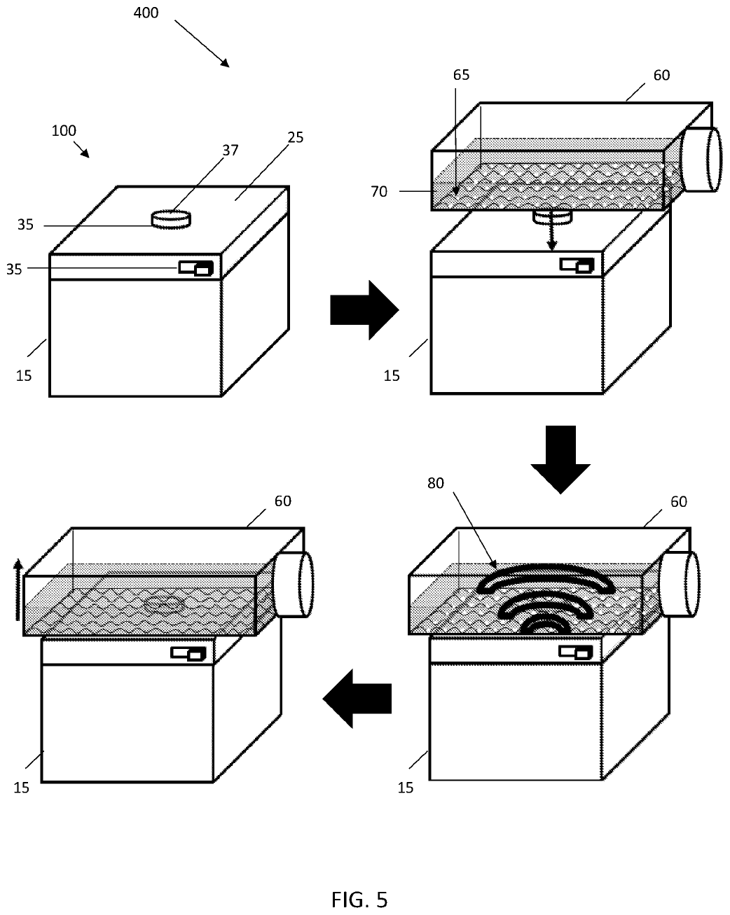

FIG. 5 shows a method 400 of using the device according to an embodiment of the invention. The ultrasonic device 100 comprises a housing 15 disposed around an ultrasonic transducer, and ultrasonic horn 35. A face 37 of the ultrasonic horn protrudes from an ultrasonic surface 25 of the housing 15. The ultrasonic device for wetting wells of microcavity substrates is in an “OFF” position prior to use. The “OFF” and “ON” modes may be designated by a power switch 35. When the device power switch 35 is turned to the “ON” state, it remains in a standby mode until activated by a cell culture container 60.

The cell culture container may be any suitable cell culture container, such as a microcavity flask, microcavity plate, multilayered microcavity cell culture vessel, or other cell culture container capable of culturing cells within microcavity wells. The cell culture container comprises a microcavity substrate comprising microcavity wells. The cell culture container may comprise a bulk spheroid microcavity substrate.

Due to the size of microcavity wells, when cell culture media 70 or other liquid is added to the cell culture container, air bubbles 65 are trapped in the microcavity wells, instead of the wells filling with cell culture media 70. In method 400, when a cell culture container 60 containing trapped air bubbles 65 is placed flush against the face 37 of the ultrasonic horn, the ultrasonic device 100 is activated and an ultrasonic waveform 80 is generated. The ultrasonic waveform 80 provides mechanical agitation to vibrate the cell culture container 60 and dislodge any trapped air bubbles 65 within the microcavity wells. The ultrasonic energy provided may be continuous or may be pulsed. The cell culture container 60 may be held against the ultrasonic surface 25 until the trapped air bubbles 65 are removed. Upon removal of the trapped air bubbles 65, the cell culture device 60 may be removed from the ultrasonic surface 25 of the ultrasonic device 100, at which time the ultrasonic waveform 80 is stopped, and the ultrasonic device 100 remains in a standby mode.

In an embodiment, an ultrasonic device of the invention comprises activation buttons or switches. The ultrasonic device for wetting wells of microcavity substrates in a cell culture container is in an “OFF” position prior to use. When the device power switch is turned to the “ON” state, it remains in a standby mode until the activation switches are depressed by a cell culture container. Depressing the activation switches activates the ultrasonic device and mechanical energy from an ultrasonic transducer is focused by an ultrasonic horn. Ultrasonic energy is applied to the cell culture container when the cell culture container is positioned or held flush against a face of the ultrasonic horn. Thus, when the cell culture container containing the bulk spheroid microcavity substrate is placed against the ultrasonic surface of the ultrasonic device to depress activation switches, and held flush against the face of the ultrasonic horn, an ultrasonic waveform is generated to vibrate the cell culture container and dislodge any trapped air bubbles within the microcavity wells. The cell culture container may be manually maneuvered while applying the ultrasonic energy so that different areas of a surface of the cell culture container are disposed flush against the face of the ultrasonic horn. Upon removal of the trapped air bubbles, the cell culture container is removed from the ultrasonic surface of the ultrasonic device, at which time the ultrasonic waveform is stopped, and the ultrasonic device remains in a standby mode.

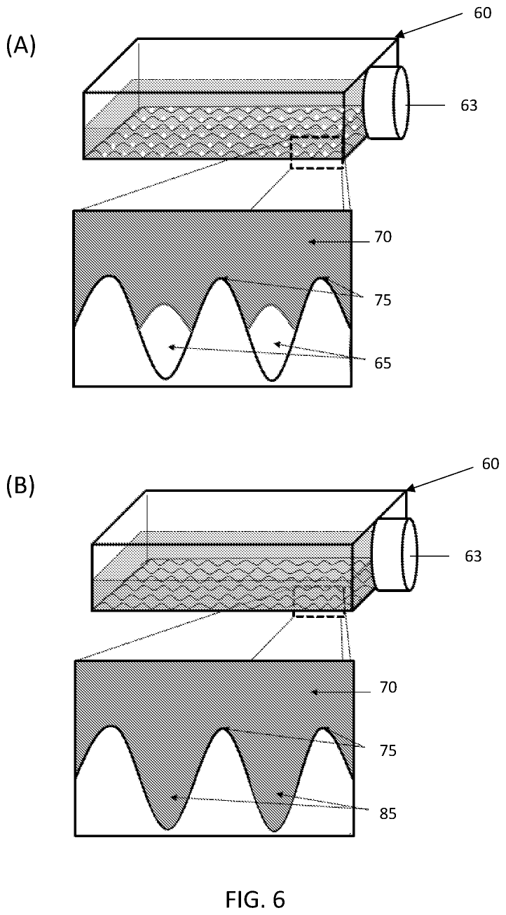

FIG. 6 shows a schematic representation of a cell culture container, such as a culture flask, before (A) undergoing the method of the invention and after (B) application of the method. As seen in view (A), the culture flask 60 may have a cap or lid 63 and contains a bulk spheroid microcavity substrate 75. Trapped air bubbles 65 form at the bottom of the microcavity wells of the microcavity substrate 75 upon addition of cell culture media 70 or other fluid used for wetting out surface of the substrate. In view (B), the culture flask 60 is shown after activation of the ultrasonic device. After activation of the device, the cavitation of the air bubbles occurs. As shown in view (B), the cavitation of the trapped air bubbles has occurred, and the microcavity substrate 75 comprises fluid filled microcavities 85. Thus, the microcavities have been wetted out and are filled with fluid prior to adding cells for spheroid formation.

Methods of releasing cell aggregates from a cell culture container are provided. For example, the cell aggregates may be spheroids or organoids. When culturing spheroids, the aggregated spheroid culture may grow too large and lodge itself in the microcavity. Methods of using the ultrasonic device of the invention dislodges the aggregated spheroid culture without damaging the spheroid, thus allowing for harvesting of the spheroids. According to an aspect of the invention, methods of releasing cell aggregates from a cell culture container comprise positioning a cell culture container at an ultrasonic surface of an ultrasonic device, the cell culture container comprising cell aggregates in microcavity wells; and releasing the cell aggregates from the microcavity wells by applying mechanical agitation from the ultrasonic device to a surface of the cell culture container.

The cell aggregates may be spheroids or organoids. The surface of the cell culture container may be a bottom surface. In some embodiments, the cell culture container is a bulk substrate for generating spheroids or organoids. In some embodiments, the cell culture container comprises a microcavity plate, microcavity flask, or multilayered microcavity cell culture vessel. In some embodiments, the cell culture container is a microcavity flask and comprises a T-25 flask, a T-75 flask, a T-175 flask, or a T-225 flask. In some embodiments, the cell culture container is a microcavity plate and comprises a 96-well spheroid microplate, a 384-well spheroid microplate, or a 1536-well spheroid microplate. In some embodiments, the cell culture container is a multilayered microcavity cell culture vessel and comprises a CellSTACK® vessel (manufactured by Corning Incorporated, Corning, N.Y.), HYPERFlask® vessel (manufactured by Corning Incorporated, Corning, N.Y.), or HYPERStack® vessel (manufactured by Corning Incorporated, Corning, N.Y.). It should be understood that other cell culture containers are contemplated and possible.

The mechanical agitation may be applied to the cell culture container in increments of 15 seconds or less. The mechanical agitation may be applied to the cell culture container continuously. The mechanical agitation may be applied to the cell culture container in pulses. In some embodiments, the ultrasonic device is pulsed in increments of hundreds of milliseconds upwards to about 15 seconds. By pulsing the ultrasonic transducer, rather than running the ultrasonic transducer continuously, methods of the invention allow for more efficient cavitation actions in clearing the microcavity wells.

In an example, the device was run continuously for about 15 seconds and then was turned off. After that point, the power switch was alternated between the ON and OFF positions. When the power switch was turned OFF, trapped bubbles were observed floating to the surface of the liquid in the cell culture vessel. The frequency at which the ultrasonic transducer operated created a point of suspended animation for the bubbles, which were released from the microcavities but kept in place within the fluid. Once the ultrasonic vibration was turned off, the bubbles escaped at the air-liquid interface.

Although multiple embodiments of the present disclosure have been illustrated in the accompanying drawings and described in the foregoing detailed description, it should be understood that the disclosure is not limited to the disclosed embodiments, but is capable of numerous rearrangements, modifications and substitutions without departing from the disclosure as set forth and defined by the following claims.

What is claimed is:

1. (canceled)

2. The kit of claim 30, wherein the ultrasonic transducer is an ultrasonic piezoelectric transducer.

3. (canceled)

4. The kit of claim 30, wherein the cell culture container comprises a microcavity plate, microcavity flask, or multilayered microcavity cell culture vessel.

5. (canceled)

6. (canceled)

7. The kit of claim 30, wherein the ultrasonic frequency is 40 kHz.

8. The kit of claim 30, wherein the ultrasonic device further comprises an ultrasonic power driver board.

9. The kit of claim 30, wherein a power switch is disposed on a surface of the housing.

10. The kit of claim 30, wherein indicator lights are disposed on a surface of the housing.

11. The kit of claim 30, further comprising activation switches on the ultrasonic surface.

12. The kit of claim 30, wherein the ultrasonic device provides continuous vibration.

13. The kit of claim 30, wherein the ultrasonic device provides pulsed vibration.

14. The kit of claim 30, wherein the ultrasonic device is portable.

15. A method of removing trapped air bubbles from a cell culture container:
positioning a cell culture container at an ultrasonic surface of an ultrasonic device, the cell culture container comprising air bubbles trapped in liquid within microcavity wells of the cell culture container; and
generating mechanical agitation at the ultrasonic surface to remove the trapped air bubbles from the microcavity wells.

16. The method of claim 15, wherein the mechanical agitation is a pulsed vibration.

17. The method of claim 15, wherein the mechanical agitation is a continuous vibration.

18. The method of claim 15, wherein generating mechanical agitation comprises using an ultrasonic horn to provide and focus ultrasonic energy from an ultrasonic transducer.

19. The method of claim 18, wherein mechanical agitation is applied to the cell culture container by holding the cell culture container against the ultrasonic horn at the ultrasonic surface.

20. The method of claim 15, wherein the cell culture container is a microcavity plate, microcavity flask, or multilayered microcavity cell culture vessel.

21. The method of claim 20, wherein the cell culture container comprises a microcavity substrate for generating spheroids or organoids.

22. The method of claim 15, wherein mechanical agitation is applied to the cell culture container for increments of 15 seconds or less.

23. A method of releasing cell aggregates from a cell culture container comprising:
positioning a cell culture container at an ultrasonic surface of an ultrasonic device, the cell culture container comprising cell aggregates in microcavity wells; and
releasing the cell aggregates from the microcavity wells by applying mechanical agitation from the ultrasonic device to a surface of the cell culture container.

24. The method of claim 23, wherein the surface of the cell culture container is a bottom surface.

25. The method of claim 23, wherein mechanical agitation is applied in increments of 15 seconds or less.

26. The method of claim 25, wherein the mechanical agitation is applied continuously.

27. The method of claim 25, wherein the mechanical agitation is applied in pulses.

28. The method of claim 23, wherein the cell aggregates are spheroids or organoids.

29. The method of claim 23, wherein the cell culture container comprises a microcavity plate, microcavity flask, or multilayered microcavity cell culture vessel.

30. A kit comprising:
an ultrasonic device comprising
a housing comprising an ultrasonic surface, the housing having an aperture;
an ultrasonic transducer disposed within the housing, the ultrasonic transducer operating at a frequency of between 25 kHz and 100 kHz; and
an ultrasonic horn having a face protruding from the aperture, the ultrasonic horn providing focused energy from the ultrasonic transducer to the ultrasonic surface; and
a cell culture container operatively connected to the ultrasonic device, the cell culture container comprising a microcavity substrate for generating spheroids or organoids.
